(19) United States
(12) Patent Application Publication
(10) Pub. No.: US 2025/0066381 A1
(43) Pub. Date: Feb. 27, 2025

(54) ONE-POT, MULTI-COMPONENT MANUFACTURING METHOD FOR TETRAHYDROTHIENOPYRIDINE DERIVATIVES INCLUDING N-SUBSTITUTED AMINO ACIDS

(71) Applicant: MOLECULES & MATERIALS, Daejeon (KR)

(72) Inventors: Kee In LEE, Daejeon (KR); So Yeon NAM, Daejeon (KR); Venkata Subbaiah SADU, Daejeon (KR); Sung Yeon HONG, Daejeon (KR)

(73) Assignee: MOLECULES & MATERIALS, Daejeon (KR)

(21) Appl. No.: 18/481,808

(22) Filed: Oct. 5, 2023

(30) Foreign Application Priority Data
Aug. 21, 2023 (KR) 10-2023-0109288

(51) Int. Cl. C07D 495/04; C07C 227/04; C07C 227/40; C07D 295/15

(52) U.S. Cl. CPC C07D 495/04; C07C 227/04; C07C 227/40; C07D 295/15

(57) ABSTRACT

Disclosed is a one-spot, multi-component manufacturing method for N-substituted amino acids using various amines and, specifically, a manufacturing method for Chemical Formula 1 and Chemical Formula 2, which are core intermediates for thienopyridine-based antiplatelet agents, wherein a synthesis method for Chemical Formulas 1 and 2, which are core intermediates for thienopyridine-based antiplatelet agents, with high yields and reduced reaction times, by a one-pot reaction in the presence of multi-component materials including glyoxylic acid, and diol/boronic acid, and/or molecular sieve/boronic acid by using tetrahydrothienopyridine derivatives as starting materials.

### CROSS-REFERENCE TO RELATED APPLICATION(S)

This application is based on and claims priority under 35 U.S.C. § 119 to Korean Patent Application No. 10-2023-0109288, filed on Aug. 21, 2023, filed in the Korean Intellectual Property Office, the disclosure of which is herein incorporated by reference in its entirety.

### BACKGROUND OF THE INVENTION

### 1. Field of the Invention

The present disclosure relates to a one-pot, multi-component manufacturing method for N-substituted amino acids using various amines.

The present disclosure provides core intermediates for thienopyridine-based oral antiplatelet agents by using a one-pot, multi-component manufacturing method for N-substituted amino acids including tetrahydrothienopyridine derivatives.

### 2. Description of the Prior Art

### 1. Petasis Borono-Mannich (PBM) Reaction

In the Petasis reaction, nitrogen-substituted amines are synthesized by multi-component reactions of an amine, a carbonyl, and a vinyl- or aryl-boronic acid as shown in Scheme 1 (Petasis, Nicos A., and rini Akritopoulou, 1993).

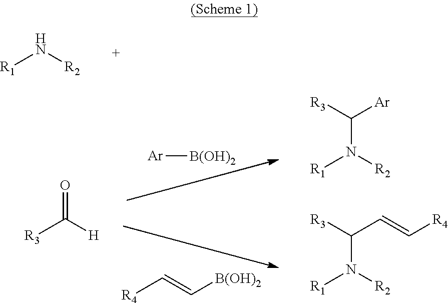

In the PBM reaction, an amino acid derivative is synthesized using glyoxylic acid as a carbonyl compound (Petasis, Nicos A. and lia A. Zavialov, 1997; Jourdan, Helene, et al., 2005).

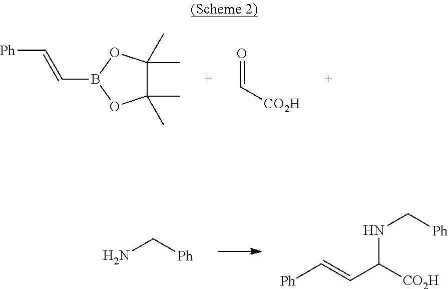 [SMILES: CC1(C)OB(/C=C/c2ccccc2)OC1(C)C ; O=CC(=O)O ; NCc1ccccc1 ; O=C(O)C(/C=C/c1ccccc1)NCc1ccccc1]

Meanwhile, there is a report on the synthesis of clopidogrel carboxylic acid, which is a clopidogrel precursor, by using glyoxylic acid, as shown in Scheme 3. However, this reaction has not been used for a clopidogrel production process to date due to a conversion rate of 30% or less even during a reaction time of one week (Kalinski, Cedric, et al., 2008).

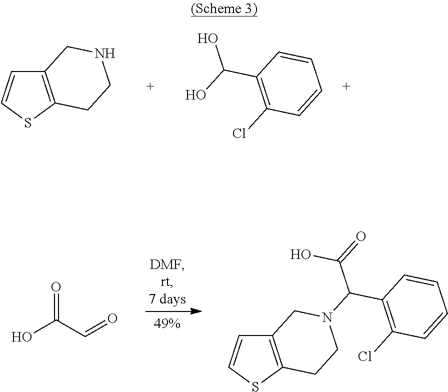 [SMILES: c1cc2c(s1)CCNC2 ; OC(O)c1ccccc1Cl ; O=CC(=O)O ; O=C(O)C(c1ccccc1Cl)N1CCc2sccc2C1]

### 2. Clopidowrel

Clopidogrel was developed by Sanofi, and the first original drug thereof was “Plavix” by Sanofi Aventis and was expired in 2009.

Clopidogrel is a prescription-based drug that is used to treat and prevent cardiovascular diseases, such as myocardial infarction, stroke, peripheral artery disease, transient ischemic attack, and (unstable) angina, caused by arteriosclerosis or thrombosis, through actions of reducing platelet aggregation and thrombus formation.

The primary cause for global mortality is cardiovascular disease, and clopidogrel is a global blockbuster antithrombotic agent.

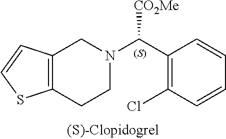

### 3. Clopidowrel Synthesis-Related Conventional Art

Sanofi's multi-step synthesis methods for clopidogrel (Patent Documents 1-6) are known wherein rac-clopidogrel was synthesized using an expensive starting material through 3 steps with a yield of 33% as shown in Scheme 4.

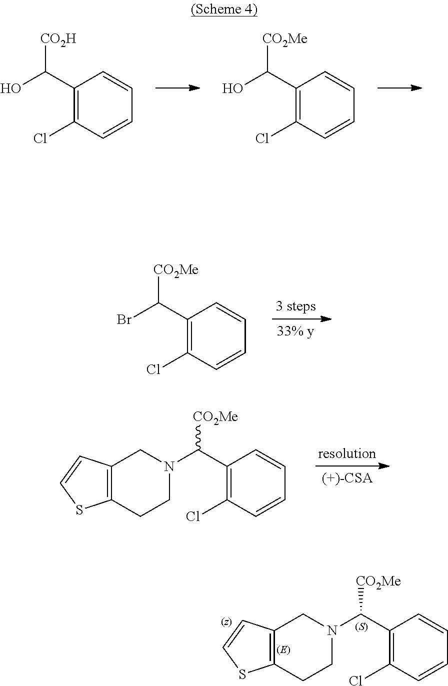

An improved method for preparing clopidogrel (Wang, Lixin, et al. 2007) is known wherein rac-clopidogrel was synthesized using a relatively inexpensive starting material through 4 steps with a yield of 5200 as shown in Scheme 5.

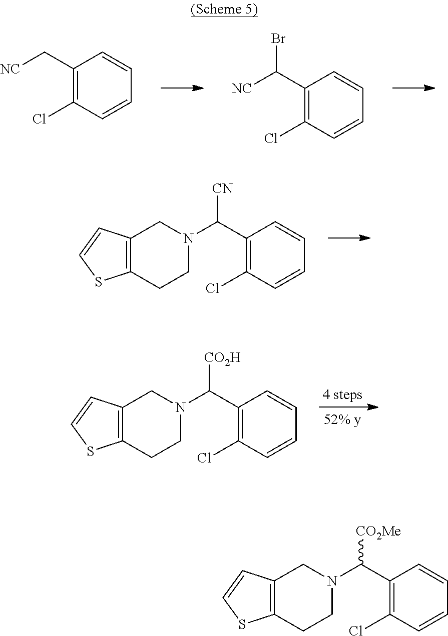 [SMILES: N#CCc1ccccc1Cl ; N#CC(Br)c1ccccc1Cl ; N#CC(c1ccccc1Cl)N1CCc2sccc2C1 ; O=C(O)C(c1ccccc1Cl)N1CCc2sccc2C1 ; COC(=O)C(c1ccccc1Cl)N1CCc2sccc2C1]

Additionally, the synthesis of (S)-clopidogrel by optical resolution of intermediates at several steps is known (Patent Document 7; and Wang, Lixin, et al., 2007).

As shown in Scheme 6 below, 2-chloromandelic acid is optically resolved by (+)-camphorsulfonic acid, 2-chloromandelic acid methyl ester is optically resolved by (+)-tartaric acid, and rac-clopidogrel is optically resolved by (−)-camphorsulfonic acid.

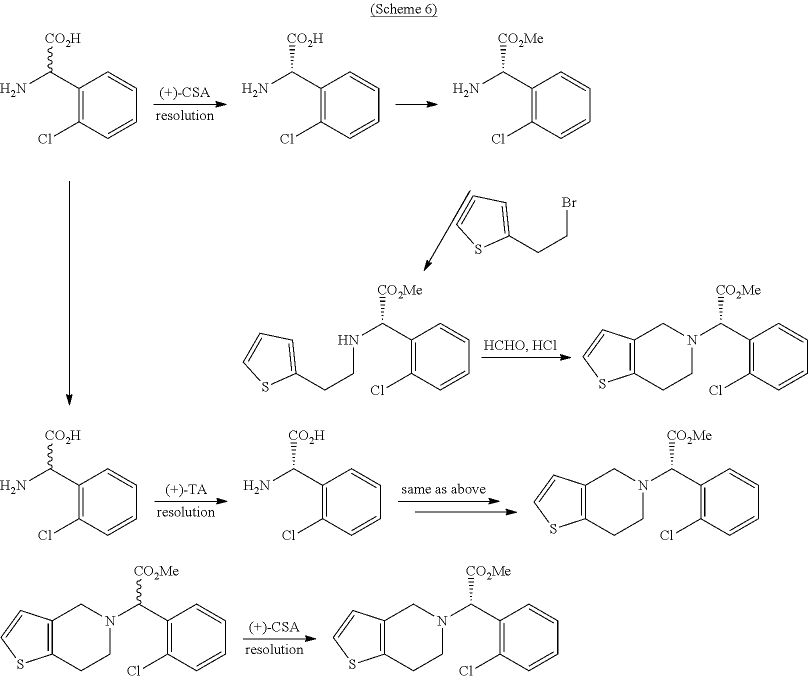

### 4. Thienopyridine-Based Antiplatelet Agent

Clopidogrel is a representative drug of the thienopyridine-based antiplatelet agents, examples of which are R-95913, prasugrel, and (S)-vicagrel, including (S)-clopidogrel.

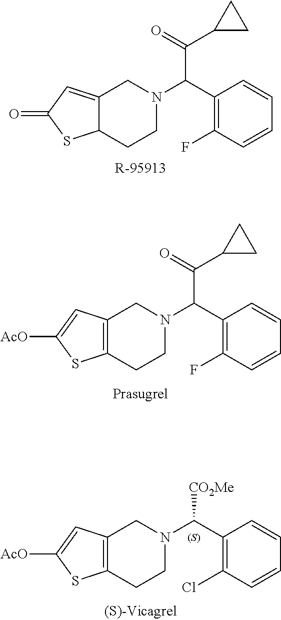

Therefore, the present inventors developed a synthesis method technically characterized by the synthesis of N-substituted amino acids through a one-pot reaction of multiple components including glyoxylic acid, catechol/boronic acid, or molecular sieve/boronic acid while using various amines as starting materials. As a result, the present disclosure has been completed by confirming the ease of manufacturing thienopyridine-based oral antiplatelet agents and an effect of high yield according to a one-pot reaction.

### PRIOR ART DOCUMENTS

### Patent Documents

FR Patent No. 2530247, NOUVEAUX DERIVES DE LA THIENO (3, 2-C) PYRIDINE, LEUR PROCEDE DE PREPARATION ET LEUR APPLICATION THERAPEUTIQUE, 20 Jan. 1984.
EP Patent No. 0281459, Dextrorotatory enantiomer of alpha-(4,5,6,7-tetrahydrothieno[3,2-c]pyrid-5-yl)(2-chlorophenyl)methyl acetate, process for its preparation and pharmaceutical compositions containing it, 26 Apr. 1995.
U.S. patent Ser. No. 04/529,596, Thieno [3,2-c]pyridine derivatives and their therapeutic application, 16 Jul. 1985.
U.S. patent Ser. No. 04/847,265, Dextro-rotatory enantiomer of methyl alpha-5 (4,5,6,7-tetrahydro (3,2-c) thieno pyridyl) (2-chlorophenyl)-acetate and the pharmaceutical compositions containing it, 11 Jul. 1989.
U.S. patent Ser. No. 05/036,156, Process for the preparation of alpha-bromo-phenylacetic acids, 30 Jul. 1991.
WO1999018110A1, HYDROXYACETIC ESTER DERIVATIVES, PREPARATION METHOD AND USE AS SYNTHESIS INTERMEDIATES, 15 Apr. 1999.
WO2008034912A, PROCESS FOR THE SYNTHESIS OF CLOPIDOGREL AND NEW FORMS OF PHARMACEUTICALLYACCEPTABLE SALTS THEREOF, 27 Mar. 2008.

### Non-Patent Documents

Jourdan, Helene, et al. “On the use of boronates in the Petasis reaction.” Tetrahedron letters 46.46 (2005): 8027-8031.
Kalinski, Cedric, et al. “Multicomponent reactions as a powerful tool for generic drug synthesis.” Synthesis (2008): 4007-4011.
Petasis, Nicos A., and rini Akritopoulou. “The boronic acid mannich reaction: Anew method for the synthesis of geometrically pure allylamines.” Tetrahedron Letters 34.4 (1993): 583-586.
Petasis, Nicos A., and Ilia A. Zavialov. “A new and practical synthesis of α-amino acids from alkenyl boronic acids.” Journal of the American Chemical Society 119.2 (1997): 445-446.
Wang, Lixin, et al. “Synthetic improvements in the preparation of clopidogrel.” Organic process research & development 11.3 (2007): 487-489.

### SUMMARY OF THE INVENTION

The present disclosure is characterized in that the improvement of yields and the reduction of reaction times are achieved by introducing the Petasis-borono-Mannich (PBM) reaction and modifying a boronic acid into a boronic ester in the PBM reaction.

The present disclosure is characterized in that in the PBM reaction, the reaction yield is maximized by adding a diol or a molecular sieve as an additive to a boronic acid.

The present disclosure is directed to a method for manufacturing N-substituted amino acids by using various amines.

An aspect of the present disclosure is to provide a method for manufacturing an N-substituted amino acid by using a tetrahydrothienopyridine and maximize the synthesis yield of clopidogrel carboxylic acid through a one-pot reaction.

Another aspect of the present disclosure is to enable the synthesis of a core intermediate for a thienopyridine-based antiplatelet agent through a one-pot reaction.

In accordance with an aspect of the present disclosure, there is provided a method for manufacturing a tetrahydrothienopyridine N-substituted amino acid compound of Chemical Formula 1:

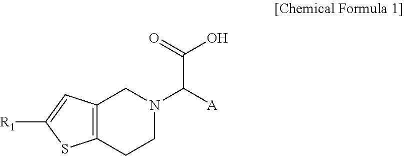

wherein in Chemical Formula 1,
A is selected from

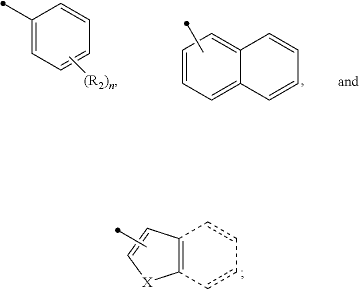

R₁ is hydrogen or an acetyl group;
R₂, at each occurrence, is independently selected from hydrogen, halogen, C₁₋₆ alkyl, C₁₋₆ alkoxy, and nitrile;
n is an integer of 0 to 2; and
the dotted lines represent the simultaneous absence or presence of bonds.

The present disclosure is directed to a method for manufacturing a tetrahydrothienopyridine compound of Chemical Formula 1, the method including: preparing a tetrahydrothienopyridine compound of Chemical Formula 1, which is an intermediate, by a one-pot reaction using, as starting materials, multi-component starting materials including a tetrahydrothienopyridine compound (1), glyoxylic acid (2), a boronic acid (3), and a diol (4), in an organic solvent in a temperature range of room temperature to 100° C. for 1 to 24 hours (first step), as shown in Scheme 7,

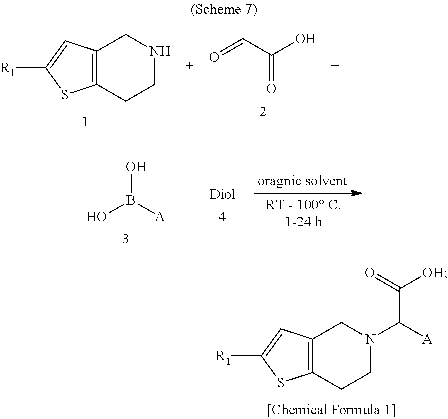

subjecting the tetrahydrothienopyridine compound of Chemical Formula 1 prepared in the first step to optical resolution by reaction with an optically active acid to form a diasteromeric salt thereof (second step).

The present disclosure is directed to a method characterized in that as shown in Scheme 8 below, a molecular sieve is additionally used or a molecular sieve is used instead of a diol (4) in the first step of Scheme 7, wherein a molecular sieve, such as Molecular Sieve 3A, 4A, or 13X, is used to maximize the reaction yield.

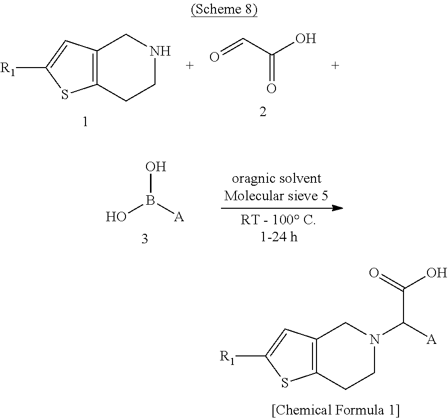

In accordance with another aspect of the present disclosure, there is provided a tetrahydrothienopyridine N-substituted amino acid compound of Chemical Formula 2:

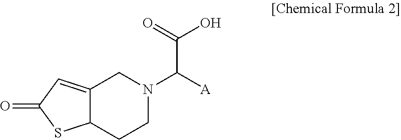

wherein, in Chemical Formula 2,
A is selected from

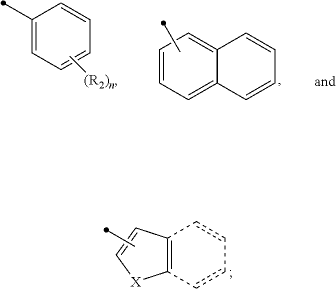

R₂, at each occurrence, is independently hydrogen or halogen;
n is an integer of 0 to 2; and
the dotted lines represent the simultaneous absence or presence of bonds.

The present disclosure is directed to a method for manufacturing a tetrahydrothienopyridine compound of Chemical Formula 2, the method including: preparing a tetrahydrothienopyridine compound of Chemical Formula 2, which is an intermediate, by a one-pot reaction using, as starting materials, multi-component starting materials including a tetrahydrothienopyridine compound (1), glyoxylic acid (2), a boronic acid (3), and a diol (4), in an organic solvent in a temperature range of room temperature to 100° C. for 1 to 24 hours (first step), as shown in Scheme 8,

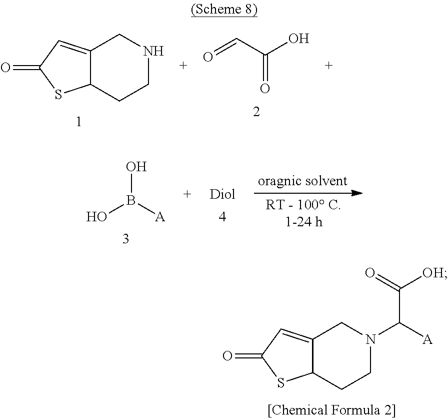

and
subjecting the tetrahydrothienopyridine compound of Chemical Formula 2 prepared in the first step to optical resolution by reaction with an optically active acid to form a diasteromeric salt thereof (second step).

The present disclosure is directed to a method characterized in that as shown in Scheme 9 below, a molecular sieve is additionally used or a molecular sieve is used instead of a diol (4) in the first step of Scheme 8, wherein a molecular sieve, such as Molecular Sieve 3A, 4A, or 13X, is used to maximize the reaction yield.

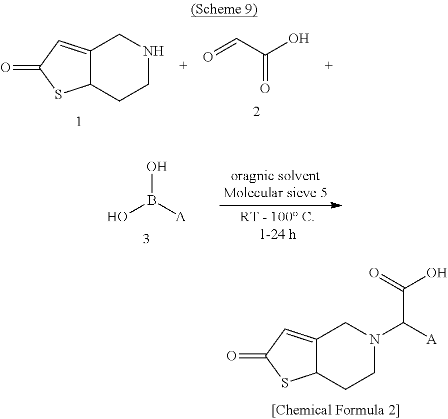

The present disclosure is directed to a method for manufacturing a tetrahydrothienopyridine N-substituted amino acid compound of Chemical Formula 1 above, wherein in Chemical Formula 1,
A is

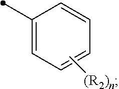

R₁ is hydrogen;
R₂, at each occurrence, is independently selected from the group consisting of hydrogen, fluoro, and chloro;
n is an integer of 0 to 2.

The present disclosure is directed to a method for manufacturing a tetrahydrothienopyridine N-substituted amino acid compound of Chemical Formula 2 above, wherein in Chemical Formula 2,
A is

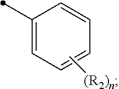

R₂, at each occurrence, is independently selected from the group consisting of hydrogen, fluoro, and chloro;
n is an integer of 0 to 2.

The diol used in the manufacturing method of the present disclosure is selected from the group consisting of ethylene glycol, 1,3-propanediol, 1,2-cyclopentanediol, 1,2-cycloheanediol, pinacol, and catechol, and of these, 1,2-diol is effective, and preferably catechol is useful.

The boronic acid (3) used in the manufacturing method of the present disclosure is selected from the group consisting of phenyl, 2-fluorophenyl, 2-chlorophenyl, 2-bromophenyl, 2-iodophenyl, 2-methylphenyl, 2-methoxyphenylboronic acid; 3-fluorophenyl, 3-chlorophenyl, 3-bromophenyl, 3-iodophenylboronic acid; 4-fluorophenyl, 4-chlorophenyl, 4-methylphenyl, 4-methoxyphenyl, 4-nitrophenyl, 4-cyanophenylboronic acid; 3-chloro-4-methylphenyl, 3,5-difluorophenyl, 3,4-(methylenedioxy)phenylboronic acid; 1-naphthyl, 2-naphtylboronic acid; 6-fluoro-3-pyridinyl, 6-chloro-3-pyridinyl, 6-bromo-3-pyridinylboronic acid; 2-furyl, 2-thienyl, 2-benzofuryl, 2-benzothienyl, 3-furyl, 3-thienyl, 3-benzofuryl, 3-benzothienylboronic acid; 2-phenylvinyl, 2-(3-fluorophenyl)vinyl, 2-(4-fluorophenyl)vinyl, and 2-(4-chlorophenyl)vinylboronic acid.

The organic solvent used in the manufacturing method of the present disclosure is at least one selected from the group consisting of DMF, DMSO, CH₂Cl₂, MeOH, EtOH, i-Pr1H, THF, Toluene, MeCN, and 1,4-Dioxane, and preferably, toluene, MeCN, and 1,4-dioxane are effective.

In the manufacturing method of the present disclosure, the reaction temperature is selected from the range of room temperature to 100° C., and a reaction time of 1 to 24 hours is selected.

The present disclosure is directed to methods for manufacturing compounds of Chemical Formula 1 and Chemical Formula 2, which are core intermediates for thienopyridine-based oral anti-platelet agents.

The present disclosure provides a manufacturing method for Chemical Formula 1 and Chemical Formula 2, which are core intermediates for thienopyridine-based antiplatelet agents, with ease of manufacturing and excellent yields through a one-pot reaction.

The present disclosure provides a synthesis method for tetrahydrothienopyridines of Chemical Formula 1 and Chemical Formula 2 with high yields through a one-pot reaction in the presence of, as starting materials, multiple components including a tetrahydrothienopyridine, glyoxylic acid, a diol, and an organic boronic acid.

The present disclosure provides a synthesis method for tetrahydrothienopyridines of Chemical Formula 1 and Chemical Formula 2 with high yields through a one-pot reaction in the presence of, as starting materials, multiple components including a tetrahydrothienopyridine, glyoxylic acid, a diol, an organic boronic acid, and a molecular sieve.

The present disclosure provides a manufacturing method for racemic clopidogrels, which are core intermediates for (S)-clopidogrel, and more specifically, a synthesis method for Chemical Formula 1 and Chemical Formula 2 with high yields through a one-pot reaction in the presence of, as starting materials, multiple components including a tetrahydrothienopyridine, glyoxylic acid, and catechol/chlorophenyl boronic acid or molecular sieve/chlorophenyl boronic acid.

Furthermore, the present disclosure provides N-substituted amino acid compounds represented by Chemical Formula 1 and Chemical Formula 2 as tetrahydrothienopyridine derivatives.

### DETAILED DESCRIPTION OF THE EXEMPLARY EMBODIMENTS

Hereinafter, preferable exemplary embodiments of the present disclosure will be described in detail. However, the present disclosure is not limited to the exemplary embodiments described herein and can be embodied in many different forms. Rather, these exemplary embodiments are provided so that the present disclosure will be thorough and complete and will fully convey the scope of the disclosure to those skilled in the art.

### Test Example 1: Exploration of Petasis Reaction Conditions

### 1-1. Solvent

In a 10-mL round flask, 4,5,6,7-tetrahydrothieno[3,2-c]pyridine (140 mg, 1.0 mmol), glyoxylic acid monohydrate (1.2 eq), 2-chlorophenylboronic acid (1.2 eq), and a solvent (3 mL) were placed and stirred at 90° C. for 4 hours. The reaction product was purified and the yield was measured. The type of solvent used and the reaction yield according to the solvent are shown in Table 1.

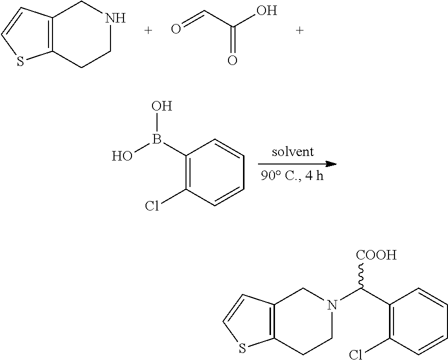 [SMILES: c1cc2c(s1)CCNC2 ; O=CC(=O)O ; OB(O)c1ccccc1Cl ; O=C(O)C(c1ccccc1Cl)N1CCc2sccc2C1]

TABLE 1

| Test Example | Solvent | Yield, % |
|---|---|---|
| 1-1 | DMF | 17% |
| 1-2 | EtOH | <5% |
| 1-3 | MeCN | 23% |
| 1-4 | 1,4-dioxane | 25% |

### 1-2. Using of Boronic Acid Ester Instead of Boronic Acid

In a 10-mL round flask, 4,5,6,7-tetrahydrothieno[3,2-c]pyridine (140 mg, 1.0 mmol), glyoxylic acid monohydrate (1.2 eq), a boronic ester (1.2 eq), and 1,4-dioxane (3 mL) were placed and stirred at 90° C. for 4 hours. The reaction product was purified and the yield was measured. The type of boronic ester used and the reaction yield according to the used boronic ester are shown in Table 2.

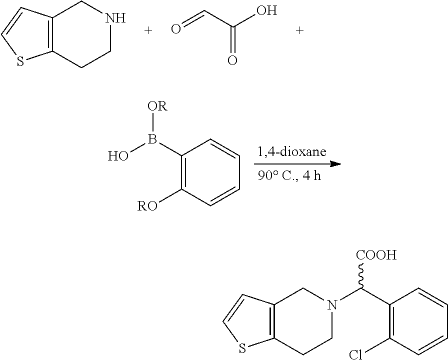

TABLE 2

| Test Example | Boronic ester | Yield, % |
|---|---|---|
| 1-5 | 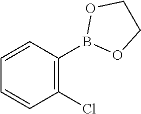 [SMILES: Clc1ccccc1B1OCCO1] | 37% |
| 1-6 | 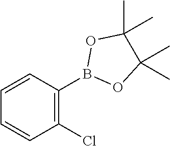 [SMILES: CC1(C)OB(c2ccccc2Cl)OC1(C)C] | <5% |
| 1-7 | 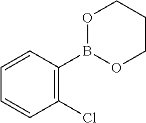 [SMILES: Clc1ccccc1B1OCCCO1] | 15% |
| 1-8 | 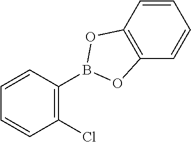 [SMILES: Clc1ccccc1B1Oc2ccccc2O1] | 87% |
| 1-9 | 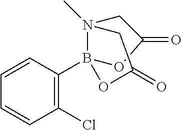 | trace |

### Test Example 2: Establishment of Diol-Modified Petasis Reaction Conditions

### 2-1. Addition of Boronic Acid and Diol Instead of Boronic Ester of Test Example 1-8

In a 10-mL round flask, 4,5,6,7-tetrahydrothieno[3,2-c]pyridine (140 mg, 1.0 mmol), glyoxylic acid monohydrate (1.2 eq), 2-chlorophenylboronic acid (1.2 eq), 1,4-dioxane (3 mL), and a diol (1.2 eq) were placed and stirred at 90° C. for 4 hours. The reaction product was purified and the yield was measured. The reaction yields according to the boronic acid and the type of diol used are shown in Table 3.

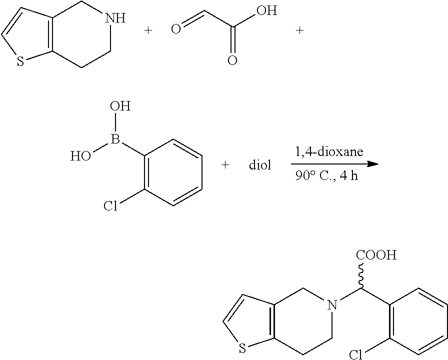 [SMILES: c1cc2c(s1)CCNC2 ; O=CC(=O)O ; OB(O)c1ccccc1Cl ; O=C(O)C(c1ccccc1Cl)N1CCc2sccc2C1]

TABLE 3

| Test Example | Diol | Yield, % |
|---|---|---|
| 2-1 | 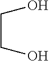 | 35% |
| 2-2 | 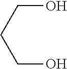 | <5% |
| 2-3 | 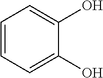 | 85% |

### 2-2. Addition of Molecular Sieve in Reaction Conditions of Test Example 2-3 (Method A)

In a 10-mL round flask, 4,5,6,7-tetrahydrothieno[3,2-c]pyridine (140 mg, 1.0 mmol), glyoxylic acid monohydrate (1.2 eq), 2-chlorophenylboronic acid (1.2 eq), 1,4-dioxane (3 mL), a diol (1.2 eq), and Molecular Sieve 3A (500 mg) were placed and stirred at 90° C. for 4 hours. The reaction product was purified and the yield was measured. The reaction yield according to the use of Molecular Sieve 3A as an additive are shown in Table 4.

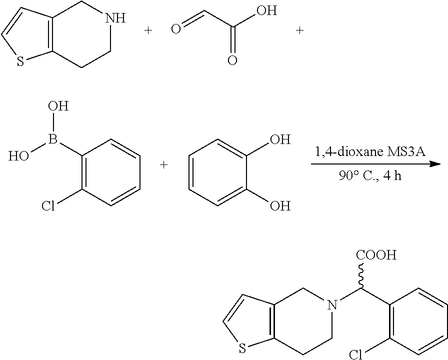 [SMILES: c1cc2c(s1)CCNC2 ; O=CC(=O)O ; OB(O)c1ccccc1Cl ; Oc1ccccc1O ; O=C(O)C(c1ccccc1Cl)N1CCc2sccc2C1]

TABLE 4

| Test Example | Reaction additive | Yield, % |
|---|---|---|
| 2-4 | 3A MS | 96% |

### Test Example 3: Establishment of Petasis Reaction Conditions in Presence of Molecular Sieve

### 3-1. Addition of Molecular Sieve in Reaction Conditions of Test Example 1-4 (Method B)

In a 10-mL round flask, 4,5,6,7-tetrahydrothieno[3,2-c]pyridine (140 mg, 1.0 mmol), glyoxylic acid monohydrate (1.2 eq), 2-chlorophenylboronic acid (1.2 eq), 1,4-dioxane (3 mL), Molecular Sieve 3A (500 mg) were placed and stirred at 90° C. for 4 hours. The reaction product was purified and the yield was measured. The reaction yield according to the use of Molecular Sieve 3A as an additive are shown in Table 5.

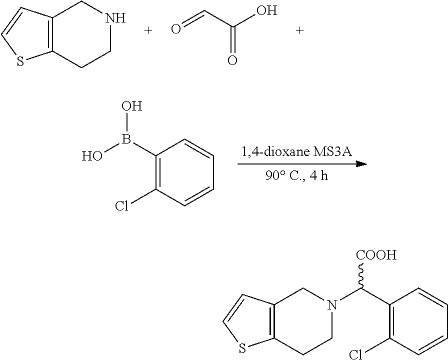 [SMILES: c1cc2c(s1)CCNC2 ; O=CC(=O)O ; OB(O)c1ccccc1Cl ; O=C(O)C(c1ccccc1Cl)N1CCc2sccc2C1]

TABLE 5

| Test Example | Reaction additive | Yield, % |
|---|---|---|
| 3-1 | 3A MS | 99% |

### Test Example 4: Manufacturing of Compounds Using Method A

Various amino acids were synthesized through a one-pot process by using several types of boronic acids and amines as described in Test Example 2-2.

Compounds synthesized by [Method A] are exemplified below. However, the compounds are not limited to the structures below.

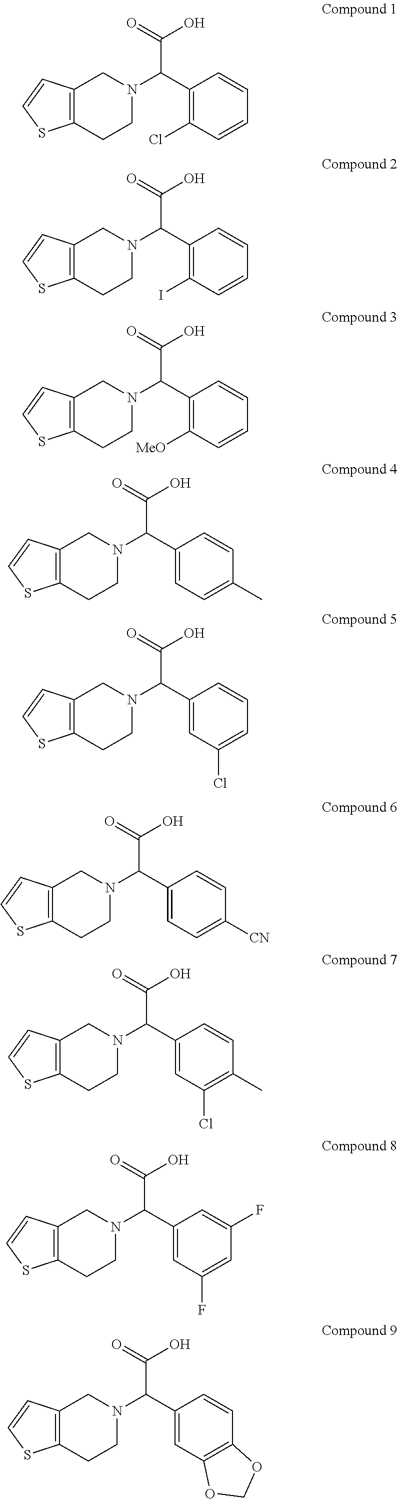

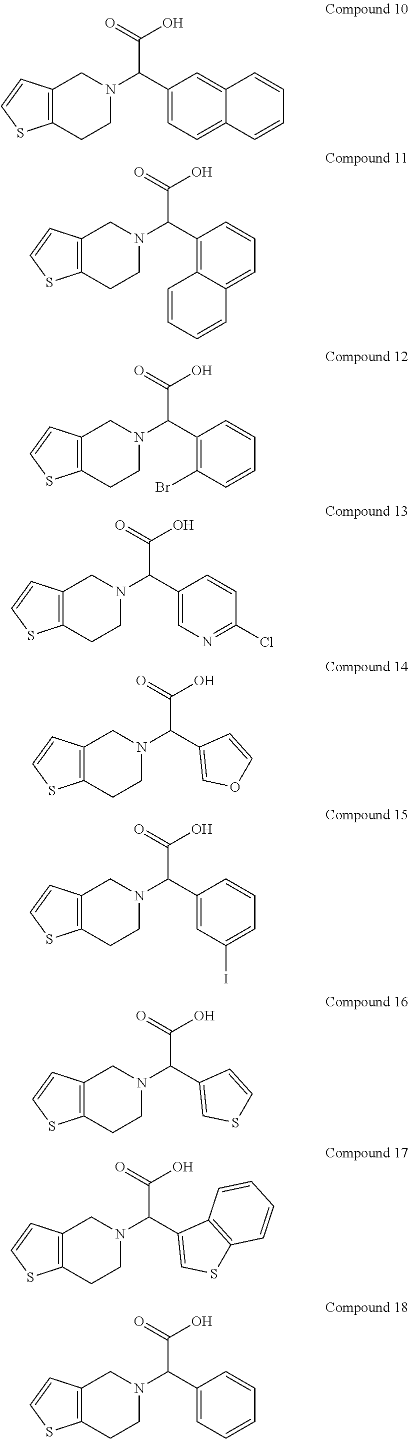

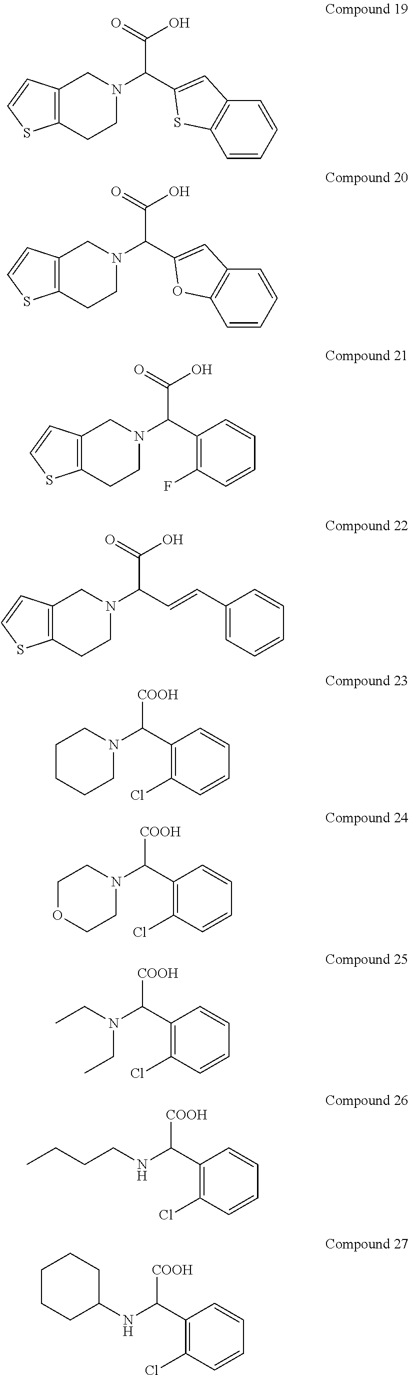

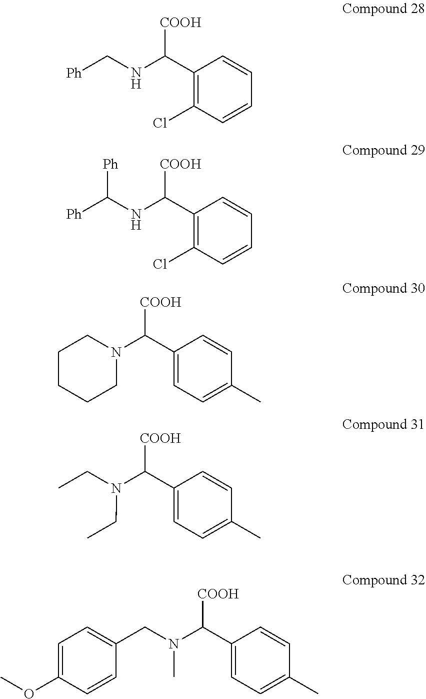

### Example 1: Synthesis of Compound 1

In a 10-mL round flask, 4,5,6,7-tetrahydrothieno[3,2-c]pyridine (139.2 mg, 1.0 mmol), glyoxylic acid monohydrate (110.5 mg, 1.2 mmol), 2-chlorophenylboronic acid (187.6 mg, 1.2 mmol), pyrocatechol (132.1 mg, 1.2 mmol), Molecular Sieve 3A (500 mg), and 1,4-dioxane (3 ml) were placed and stirred at 90° C. for 4 hours. The reaction mixture was filtered through celite, and then the organic layer was extracted by addition of CH₂Cl₂ and water. The organic layer was dried over MgSO₄ and then filtered, and the filtrate thus obtained was distilled under reduced pressure to remove the solvent. The residue was purified by SiO₂ column chromatography (5% MeOH/MC) to give Compound 1 (296 mg, yield: 96%)

¹H NMR (400 MHz, DMSO) δ 7.70 (d, 1H), 7.45 (d, 1H), 7.33 (t, 2H), 7.26 (d, 1H), 6.76 (d, 1H), 4.59 (s, 1H), 3.68 (d, 1H), 3.55 (d, 1H), 2.85 (d, 1H), 2.75 (s, 3H).

### Example 2: Synthesis of Compound 2

In a 10-mL round flask, 4,5,6,7-tetrahydrothieno[3,2-c]pyridine (139.2 mg, 1 mmol), glyoxylic acid monohydrate (110.5 mg, 1.2 mmol), 2-iodinephenylboronic acid (297.4 mg, 1.2 mmol), pyrocatechol (132.1 mg, 1.2 mmol), Molecular Sieve 3A (500 mg), and 1,4-dioxane (3 ml) were placed and stirred at 90° C. for 4 hours. The reaction mixture was filtered through celite, and then the organic layer was extracted by addition of CH₂Cl₂ and water. The organic layer was dried over MgSO₄ and then filtered, and the filtrate thus obtained was distilled under reduced pressure to remove the solvent. The residue was purified by SiO₂ column chromatography (5% MeOH/MC) to give Compound 2 (372 mg, yield: 93%) ¹H NMR (400 MHz, DMSO) δ 7.87 (d, 1H), 7.64 (d, 1H), 7.39 (t, 1H), 7.25 (d, 1H), 7.04 (t, 1H), 6.75 (d, 1H), 4.40 (s, 1H), 3.63 (t, 2H), 2.86 (dd, 1H), 2.78 (dd, 1H), 2.72 (s, 2H).

### Example 3: Synthesis of Compound 3

In a 10-mL round flask, 4,5,6,7-tetrahydrothieno[3,2-c]pyridine (139.2 mg, 1 mmol), glyoxylic acid monohydrate (110.5 mg, 1.2 mmol), (2-methoxyphenyl)boronic acid (182.4 mg, 1.2 mmol), pyrocatechol (132.1 mg, 1.2 mmol), Molecular Sieve 3A (500 mg), and 1,4-dioxane (3 ml) were placed and stirred at 90° C. for 4 hours. The reaction mixture was filtered through celite, and then the organic layer was extracted by addition of CH₂Cl₂ and water. The organic layer was dried over MgSO₄ and then filtered, and the filtrate thus obtained was distilled under reduced pressure to remove the solvent. The residue was purified by SiO₂ column chromatography (5% MeOH/MC) to give Compound 3 (334 mg, yield: 99%)

¹H NMR (400 MHz, DMSO) δ 7.45 (d, 1H), 7.35-7.28 (m, 1H), 7.26 (d, 1H), 7.05 (d, 1H), 6.97 (t, 1H), 6.77 (d, 1H), 4.66 (s, 1H), 3.79 (s, 3H), 3.61 (q, 2H), 2.91-2.70 (m, 4H).

### Example 4: Synthesis of Compound 4

By the same method as in Example 1, p-tolylboronic acid (163.2 mg, 1.2 mmol) was used to give Compound 4 (279 mg, yield: 97%)

¹H NMR (400 MHz, DMSO) δ 7.34 (d, 2H), 7.24 (d, 1H), 7.11 (d, 2H), 6.71 (d, 1H), 3.98 (s, 1H), 3.54 (s, 1H), 3.46 (d, 1H), 2.73 (s, 4H), 2.28 (s, 3H).

### Example 5: Synthesis of Compound 5

By the same method as in Example 1, 2-chlorophenylboronic acid (187.6 mg, 1.2 mmol) was used to give Compound 5 (295 mg, yield: 96%)

¹H NMR (400 MHz, DMSO) δ 7.52 (s, 1H), 7.46-7.29 (m, 3H), 7.26 (d, 1H), 6.75 (d, 1H), 4.13 (s, 1H), 3.59 (d, 1H), 3.48 (d, 1H), 2.74 (d, 4H).

### Example 6: Synthesis of Compound 6

By the same method as in Example 1, (4-cyanophenyl)boronic acid (176.3 mg, 1.2 mmol) was used to give Compound 6 (50 mg, yield: 17%)

¹H NMR (400 MHz, DMSO) δ 7.75 (d, 2H), 7.66 (d, 2H), 7.24 (d, 1H), 6.71 (d, 1H), 4.03 (s, 1H), 3.68-3.56 (m, 1H), 3.43 (d, 1H), 2.72 (dd, 4H).

### Example 7: Synthesis of Compound 7

By the same method as in Example 1, 3-chloro-4-methylphenylboronic acid (204.5 mg, 1.2 mmol) was used to give Compound 7 (318 mg, yield: 99%)

¹H NMR (400 MHz, DMSO) δ 7.49 (s, 1H), 7.31 (s, 2H), 7.26 (d, 1H), 6.76 (d, 1H), 4.11 (s, 1H), 3.57 (d, 1H), 3.48 (d, 1H), 2.75 (s, 4H), 2.32 (s, 3H).

### Example 8: Synthesis of Compound 8

By the same method as in Example 1, (3,5-difluorophenyl)boronic acid (189.5 mg, 1.2 mmol) was used to give Compound 8 (258 mg, yield: 83%)

¹H NMR (400 MHz, DMSO) δ 7.26 (d, 1H), 7.15 (t, 3H), 6.76 (d, 1H), 4.16 (s, 1H), 3.62 (d, 1H), 3.48 (d, 1H), 2.75 (s, 4H).

### Example 9: Synthesis of Compound 9

By the same method as in Example 1, benzo[d][1,3]dioxol-5-ylboronic acid (199.1 mg, 1.2 mmol) was used to give Compound 9 (278 mg, yield: 88%)

¹H NMR (400 MHz, DMSO) δ 7.26 (d, 1H), 7.02 (s, 1H), 6.89 (q, 2H), 6.76 (d, 1H), 6.01 (d, 2H), 4.03 (s, 1H), 3.57 (d, 1H), 3.47 (d, 1H), 2.75 (s, 4H).

### Example 10: Synthesis of Compound 10

By the same method as in Example 1, naphthalen-2-ylboronic acid (206.4 mg, 1.2 mmol) was used to give Compound 10 (325 mg, yield: 99%)

¹H NMR (400 MHz, DMSO) δ 7.99-7.82 (m, 4H), 7.67 (d, 1H), 7.50 (dd, 2H), 7.25 (d, 1H), 6.73 (d, 1H), 4.29 (s, 1H), 3.62 (d, 1H), 3.53 (d, 1H), 2.89-2.68 (m, 4H).

### Example 11: Synthesis of Compound 11

By the same method as in Example 1, naphthalen-1-ylboronic acid (206.4 mg 1.2 mmol) was used to give Compound 11 (325 mg yield: 99%)

¹H NMR (400 MHz, DMSO) δ 8.57 (s, 1H), 8.00-7.85 (m, 2H), 7.69 (d, 1H), 7.56-7.42 (m, 3H), 7.23 (d, 1H), 6.71 (d, 1H), 4.93 (s, 1H), 3.72-3.60 (m, 2H), 2.94-2.81 (m, 2H), 2.81-2.63 (m, 2H).

### Example 12: Synthesis of Compound 12

By the same method as in Example 1, 2-bromophenylboronic acid (241 mg 1.2 mmol) was used to give Compound 12 (307 mg yield: 87%)

¹H NMR (400 MHz, DMSO) δ 7.70 (d, 1H), 7.62 (d, 1H), 7.38 (t, 1H), 7.29-7.19 (m, 2H), 6.75 (d, 1H), 4.56 (s, 1H), 3.67 (d, 1H), 3.56 (d, 1H), 2.85 (d, 1H), 2.82-2.70 (m, 3H).

### Example 13: Synthesis of Compound 13

By the same method as in Example 1, 2-chloropyridine-5-boronic acid (188.8 mg 1.2 mmol) was used to give Compound 13 (239 mg yield: 77%)

¹H NMR (400 MHz, DMSO) δ 8.43 (d, 1H), 7.93 (dd, 1H), 7.47 (d, 1H), 7.25 (d, 1H), 6.75 (d, 1H), 4.12 (s, 1H), 3.63 (d, 1H), 3.48 (d, 1H), 2.75 (s, 4H).

### Example 14: Synthesis of Compound 14

By the same method as in Example 1, 3-furylboronic acid (134.3 mg, 1.2 mmol) was used to give Compound 14 (265 mg, yield: 99%)

¹H NMR (400 MHz, DMSO) δ 7.65 (d, 2H), 7.26 (d, 1H), 6.77 (d, 1H), 6.51 (s, 1H), 4.23 (s, 1H), 3.62 (s, 2H), 2.94-2.69 (m, 4H).

### Example 15: Synthesis of Compound 15

By the same method as in Example 1, 3-iodinephenylboronic acid (297.5 mg, 1.2 mmol) was used to give Compound 15 (296 mg yield: 74%)

¹H NMR (400 MHz, DMSO) δ 7.89 (d, 1H), 7.63 (d, 1H), 7.37 (t, 1H), 7.24 (d, 1H), 6.98 (t, 1H), 6.73 (d, 1H), 4.41 (s, 1H), 3.62 (t, 2H), 2.86 (dd, 1H), 2.81 (dd, 1H), 2.70 (s, 2H).

### Example 16: Synthesis of Compound 16

By the same method as in Example 1, 3-thipheneboronic acid (153.5 mg, 1.2 mmol) was used to give Compound 16 (193 mg, yield: 69%)

¹H NMR (400 MHz, DMSO) δ 7.46 (d, 2H), 7.25 (d, 1H), 7.17 (d, 1H), 6.75 (s, 1H), 4.29 (s, 1H), 3.59 (s, 2H), 2.78 (d, 4H).

### Example 17: Synthesis of Compound 17

By the same method as in Example 1, benzo[b]thiophen-3-ylboronic acid (213.6 mg 1.2 mmol) was used to give Compound 17 (134 mg yield: 41%)

¹H NMR (400 MHz, DMSO) δ 8.24 (s, 1H), 8.01-7.89 (m, 1H), 7.69 (s, 1H), 7.39-7.27 (m, 2H), 7.23 (d, 1H), 6.71 (s, 1H), 4.58 (s, 1H), 3.66 (s, 2H), 2.86 (s, 2H), 2.70 (s, 2H).

### Example 18: Synthesis of Compound 18

By the same method as in Example 1, pheneylboronic acid (146.3 mg, 1.2 mmol) was used to give Compound 18 (219 mg, yield: 80%)

¹H NMR (400 MHz, DMSO) δ 7.46 (d, 2H), 7.30 (ddd, 4H), 6.74 (d, 1H), 4.12 (s, 1H), 3.59 (d, 1H), 3.49 (d, 1H), 2.75 (s, 4H).

### Example 19: Synthesis of Compound 19

By the same method as in Example 1, benzo[b]thiophen-2-ylboronic acid (213.6 mg 1.2 mmol) was used to give Compound 19 (280 mg yield: 85%)

¹H NMR (400 MHz, DMSO) δ 7.88 (d, 1H), 7.77 (d, 1H), 7.41 (s, 1H), 7.37-7.23 (m, 3H), 6.76 (d, 1H), 4.61 (s, 1H), 3.72 (d, 2H), 2.97-2.85 (m, 2H), 2.77 (s, 2H).

### Example 20: Synthesis of Compound 20

By the same method as in Example 1, benzofuran-2-ylboronic acid (194.3 mg, 1.2 mmol) was used to give Compound 20 (165 mg yield: 53%)

¹H NMR (400 MHz, DMSO) δ 7.55 (d, 1H), 7.49 (d, 1H), 7.28-7.14 (m, 3H), 6.82 (s, 1H), 6.71 (s, 1H), 4.42 (s, 1H), 3.81 (d, 1H), 3.65 (d, 1H), 3.07-2.96 (m, 1H), 2.96-2.87 (m, 1H), 2.75 (s, 2H).

### Example 21: Synthesis of Compound 21

By the same method as in Example 1, (2-fluorophenyl)boronic acid (167.8 mg, 1.2 mmol) was used to give Compound 21 (291 mg yield: 99%)

¹H NMR (400 MHz, DMSO) δ 7.57 (t, 1H), 7.42-7.31 (m, 1H), 7.22 (ddd, 3H), 6.76 (d, J=5.1 Hz, 1H), 4.56 (s, 1H), 3.61 (s, 2H), 2.79 (dd, 4H).

### Example 22: Synthesis of Compound 22

By the same method as in Example 1, (E)-styrylboronic acid (177.6 mg 1.2 mmol) was used to give Compound 22 (170 mg, yield: 56%)

¹H NMR (400 MHz, DMSO) δ 7.43 (d, 2H), 7.32 (t, 2H), 7.25 (dd, 3H), 6.83-6.74 (m, 1H), 6.61 (d, 1H), 6.44 (d, 1H), 3.78 (s, 2H), 3.68 (d, 3H), 2.90 (dd, 2H), 2.83-2.67 (m, 5H).

### Example 23: Synthesis of Compound 23

In a 10-mL round flask, piperidine (85.3 mg 1.0 mmol), glyoxylic acid monohydrate (110.5 mg, 1.2 mmol), 2-chlorophenylboronic acid (187.6 mg, 1.2 mmol), pyrocatechol (132.1 mg, 1.2 mmol), Molecular Sieve 3A (500 mg), and 1,4-dioxane (3 ml) were placed and stirred at 90° C. for 4 hours. The reaction mixture was filtered through celite, and then the organic layer was extracted by addition of CH₂Cl₂ and water. The organic layer was dried over MgSO₄ and then filtered, and the filtrate thus obtained was distilled under reduced pressure to remove the solvent. The residue was purified by SiO₂ column chromatography (5% MeOH/MC) to give Compound 23 (yield: 81%).

¹H NMR (500 MHz, DMSO): δ_{H} 7.68-7.66 (m, 1H), 7.50-7.48 (m, 1H), 7.41-7.35 (m, 2H), 4.54 (s, 1H), 2.86-2.79 (m, 2H), 2.62-2.58 (m, 2H), 1.65-1.58 (m, 4H), 1.47-1.44 (m, 2H).

### Example 24: Synthesis of Compound 24

By the same method as in Example 23, morpholine (1.0 mmol) was used to give Compound 24 with a yield of 77%.

¹H NMR (500 MHz, CDCl₃): δ_{H} 7.67-7.65 (m, 1H), 7.45-7.43 (m, 1H), 7.32-7.27 (m, 2H), 5.12 (s, 1H), 3.82-3.79 (m, 4H), 3.16-3.12 (m, 2H), 2.96-2.93 (m, 2H).

### Example 25: Synthesis of Compound 25

By the same method as in Example 23, diethylamine (1.0 mmol) was used to give Compound 25 with a yield of 69%.

¹H NMR (500 MHz, CDCl₃): δ_{H} 7.65-7.63 (m, 1H), 7.37-7.32 (m, 1H), 7.24-7.21 (m, 2H), 3.84 (s, 1H), 2.72-2.70 (m, 4H), 1.11 (t, J=7.1 Hz, 6H).

### Example 26: Synthesis of Compound 26

By the same method as in Example 23, n-butylamine (1.0 mmol) was used to give Compound 26 with a yield of 17%.

¹H NMR (500 MHz, DMSO): δ_{H} 7.67-7.61 (m, 1H), 7.52-7.48 (m, 1H), 7.40-7.32 (m, 2H), 4.56 (s, 1H), 2.78 (t, J=7.2 Hz, 2H), 1.52-1.43 (m, 2H), 1.32-1.27 (m, 2H), 0.84 (t, J=7.2 Hz, 3H).

### Example 27: Synthesis of Compound 27

By the same method as in Example 23, cyclohexylamine (1.0 mmol) was used to give Compound 27 with a yield of 27%.

¹H NMR (500 MHz, DMSO): δ_{H} 7.67-7.64 (m, 1H), 7.51-7.46 (m, 1H), 7.41-7.36 (m, 2H), 4.72 (s, 1H), 2.72-2.68 (m, 1H), 1.90-1.78 (m, 2H), 1.74-1.54 (m, 3H), 1.26-1.06 (m, 5H).

### Example 28: Synthesis of Compound 28

By the same method as in Example 23, benzylamine (1.0 mmol) was used to give Compound 28 with a yield of 31%.

¹H NMR (500 MHz, CDCl₃): δ_{H} 7.41-7.19 (m, 9H), 4.57 (s, 1H), 3.93 (s, 2H).

### Example 29: Synthesis of Compound 29

By the same method as in Example 23, benzhydrylamine (1.0 mmol) was used to give Compound 29 with a yield of 62%.

¹H NMR (500 MHz, CDCl₃): δ_{H} 7.97-7.95 (m, 1H), 7.38-7.24 (m, 13H), 4.84 (s, 1H), 4.66 (s, 1H).

### Example 30: Synthesis of Compound 30

In a 10-mL round flask, piperidine (85.3 mg, 1.0 mmol), glyoxylic acid monohydrate (110.5 mg, 1.2 mmol), p-tolylboronic acid (163.2 mg, 1.2 mmol), pyrocatechol (132.1 mg, 1.2 mmol), Molecular Sieve 3 Å (500 mg), and 1,4-dioxane (3 ml) were placed and stirred at 90° C. for 4 hours. The reaction mixture was filtered through celite, and then the organic layer was extracted by addition of CH₂Cl₂ and water. The organic layer was dried over MgSO₄ and then filtered, and the filtrate thus obtained was distilled under reduced pressure to remove the solvent. The residue was purified by SiO₂ column chromatography (5% MeOH/MC) to give Compound 30 with a yield of 87%

¹H NMR (500 MHz, CDCl₃): δ_{H} 7.26 (d, J=7.7 Hz, 2H), 7.01 (d, J=7.6 Hz, 2H), 4.30 (s, 1H), 2.84 (m, 2H), 2.20 (s, 3H), 1.68 (m, 4H), 1.36 (m, 2H).

### Example 31: Synthesis of Compound 31

By the same method as in Example 30, dimethylamine (1.0 mmol) was used to give Compound 31 with a yield of 74%.

¹H NMR (500 MHz, CDCl₃): δ_{H} 7.23 (d, J=7.9 Hz, 2H), 6.96 (d, J=7.9 Hz, 2H), 4.38 (s, 1H), 3.06-2.62 (m, 4H), 2.18 (s, 3H), 0.97 (t, J=7.3 Hz, 6H).

### Example 32: Synthesis of Compound 32

By the same method as in Example 30, 4-methoxybenzylamine was used to give Compound 32 with a yield of 41%.

(500 MHz, DMSO): δ_{H} 7.35-7.26 (m, 2H), 7.20 (d, J=7.7 Hz, 2H), 7.11-7.01 (m, 2H), 6.80-6.68 (m, 2H), 4.49 (s, 1H), 3.73 (s, 3H), 3.61 (s, 2H), 3.32 (s, 3H), 2.29 (s, 3H).

### Example B: Manufacturing of Compounds Using Method B

Various amino acids were synthesized through a one-pot process by using several types of boronic acids and amines as described in Test Example 3-1.

Compounds synthesized by [Method B] are exemplified below. However, the compounds are not limited to the structures below.

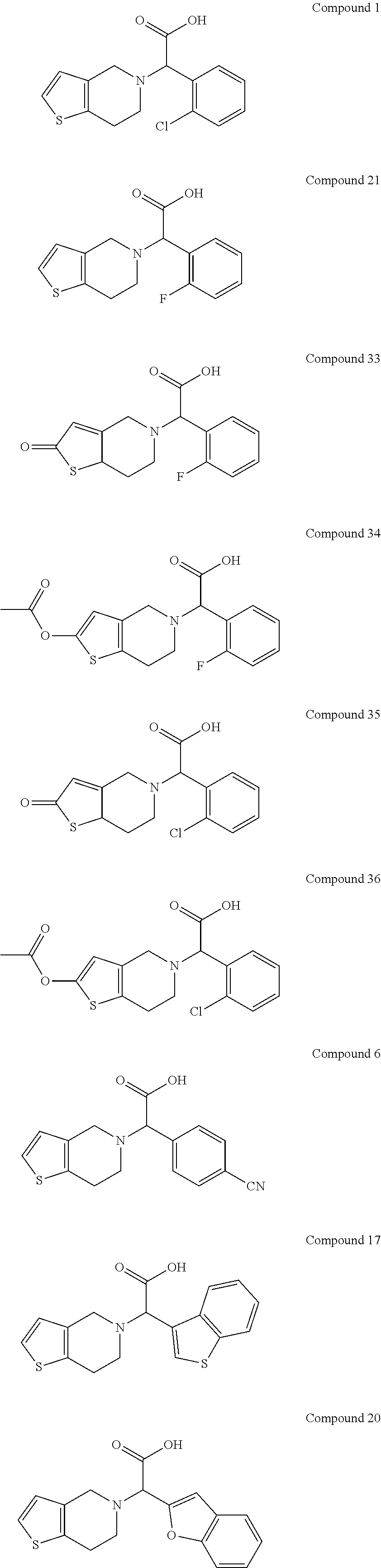

### Example 33 Synthesis of Compound 1

In a 10-mL round flask, 4,5,6,7-tetrahydrothieno[3,2-c]pyridine (139.2 mg, 1.0 mmol), glyoxylic acid monohydrate (110.5 mg, 1.2 mmol), 2-chlorophenylboronic acid (187.6 mg, 1.2 mmol), Molecular Sieve 3A (500 mg), and 1,4-dioxane (3 ml) were placed and stirred at 90° C. for 4 hours. The reaction mixture was filtered through celite, and then the organic layer was extracted by addition of CH₂Cl₂ and water. The organic layer was dried over MgSO₄ and then filtered, and the filtrate thus obtained was distilled under reduced pressure to remove the solvent. The residue was purified by SiO₂ column chromatography (50% MeOH/MC) to give Compound 1 with a yield of 99%

### Example 34: Synthesis of Compound 21

By the same method as in Example 30, (2-fluorophenyl)boronic acid (167.8 mg, 1.2 mmol) was used to give Compound 21 with a yield of 99.

### Example 35: Synthesis of Compound 33

In a 10-mL round flask, 5,6,7,7a-tetrahydrothieno[3,2-c]pyridine-2(4H)-one hydrochloride (191.7 mg, 1.0 mmol), glyoxylic acid monohydrate (110.5 mg, 1.2 mmol), (2-fluorophenyl)boronic acid (167.8 mg, 1.2 mmol), Molecular Sieve 3A (500 mg), and 1,4-dioxane (3 ml) were placed and stirred at 90° C. for 4 hours. The reaction mixture was filtered through celite, and then the organic layer was extracted by addition of CH₂Cl₂ and water. The organic layer was dried over MgSO₄ and then filtered, and the filtrate thus obtained was distilled under reduced pressure to remove the solvent. The residue was purified by SiO₂ column chromatography (5% MeOH/MC) to give Compound 33 with a yield of 40%

¹H NMR (400 MHz, DMSO) δ 7.47 (s, 1H), 7.38 (dd, 1H), 7.22 (dd, 2H), 6.18 (s, 1H), 4.57 (d, 1H), 4.46 (d, 1H), 3.94 (t, 1H), 3.22-3.09 (m, 1H), 3.02 (t, 1H), 2.57 (d, 1H), 2.40 (t, 1H), 1.59 (dd, 1H).

### Example 36: Synthesis of Compound 33

In a 10-mL round flask, 5,6,7,7a-tetrahydrothieno[3,2-c]pyridine-2(4H)-one hydrochloride (191.7 mg, 1.0 mmol), glyoxylic acid monohydrate (110.5 mg, 1.2 mmol), (2-fluorophenyl)boronic acid (167.8 mg, 1.2 mmol), Molecular Sieve 3A (500 mg), and 1,4-dioxane (3 ml) were placed and stirred at room temperature for 24 hours. The reaction mixture was filtered through celite, and then the organic layer was extracted by addition of CH₂Cl₂ and water. The organic layer was dried over MgSO₄ and then filtered, and the filtrate thus obtained was distilled under reduced pressure to remove the solvent. The residue was purified by SiO₂ column chromatography (5% MeOH/MC) to give Compound 33 with a yield of 55%

### Example 37: Synthesis of Compound 34

By the same method as in Example 36, 4,5,6,7-tetrahydrothieno[3,2-c]pyridin-2-yl acetate hydrochloride (233.2 mg, 1.0 mmol) was used to give Compound 34 with a yield of 69%.

¹H NMR (400 MHz, DMSO) δ 7.42-7.31 (m, 1H), 7.23 (ddd, 3H), 6.77 (d, J=5.1 Hz, 1H), 4.62 (s, 1H), 3.62 (s, 2H), 2.78 (dd, 4H), 2.08 (s, 3H).

### Example 38: Synthesis of Compound 35

In a 10-mL round flask, 5,6,7,7a-tetrahydrothieno[3,2-c]pyridine-2(4H)-one hydrochloride (191.7 mg, 1.0 mmol), glyoxylic acid monohydrate (110.5 mg, 1.2 mmol), 2-chlorophenylboronic acid (187.6 mg, 1.2 mmol), Molecular Sieve 3A (500 mg), and 1,4-dioxane (3 ml) were placed and stirred at room temperature for 24 hours. The reaction mixture was filtered through celite, and then the organic layer was extracted by addition of CH₂Cl₂ and water. The organic layer was dried over MgSO₄ and then filtered, and the filtrate thus obtained was distilled under reduced pressure to remove the solvent. The residue was purified by SiO₂ column chromatography (5% MeOH/MC) to give Compound 35 with a yield of 60%

¹H NMR (400 MHz, DMSO) δ 7.52 (s, 1H), 7.40 (dd, 1H), 7.23 (dd, 2H), 6.19 (s, 1H), 4.58 (d, 1H), 4.49 (d, 1H), 3.92 (t, 1H), 3.22-3.12 (m, 1H), 3.12 (t, 1H), 2.58 (d, 1H), 2.45 (t, 1H), 1.62 (dd, 1H).

### Example 39: Synthesis of Compound 36

By the same method as in Example 38, 4,5,6,7-tetrahydrothieno[3,2-c]pyridin-2-yl acetate hydrochloride (233.2 mg, 1.0 mmol) was used to give Compound 36 with a yield of 72%.

¹H NMR (400 MHz, DMSO) δ 7.40-7.28 (m, 1H), 7.21 (ddd, 3H), 6.79 (d, J=5.2 Hz, 1H), 4.65 (s, 1H), 3.61 (s, 2H), 2.77 (dd, 4H), 2.10 (s, 3H).

### Example 40: Synthesis of Compound 6

In a 10-mL round flask, 4,5,6,7-tetrahydrothieno[3,2-c]pyridine (139.2 mg, 1.0 mmol), glyoxylic acid monohydrate (110.5 mg, 1.2 mmol), (4-cyanophenyl)boronic acid (176.3 mg, 1.2 mmol), Molecular Sieve 3A (500 mg), and 1,4-dioxane (3 ml) were placed and stirred at room temperature for 4 hours. The reaction mixture was filtered through celite, and then the organic layer was extracted by addition of CH₂Cl₂ and water. The organic layer was dried over MgSO₄ and then filtered, and the filtrate thus obtained was distilled under reduced pressure to remove the solvent. The residue was purified by SiO₂ column chromatography (5% MeOH/MC) to give Compound 6 (yield: 46%).

### Example 41: Synthesis of Compound 17

By the same method as in Example 40, benzo[b]thiophen-3-ylboronic acid (213.6 mg, 1.2 mmol) was used to give Compound 17 (yield: 65%).

### Example 42: Synthesis of Compound 20

By the same method as in Example 40, benzofuran-2-ylboronic acid (194.3 mg, 1.2 mmol) was used to give Compound 20 (yield: 99%).

1. A method for manufacturing a tetrahydrothienopyridine compound of Chemical Formula 1, by a one-pot reaction of multi-component starting materials comprising a tetrahydrothienopyridine compound (1), glyoxylic acid (2), a boronic acid (3), and a diol (4) in an organic solvent in a temperature range of room temperature to 100° C. for 1-24 hours: 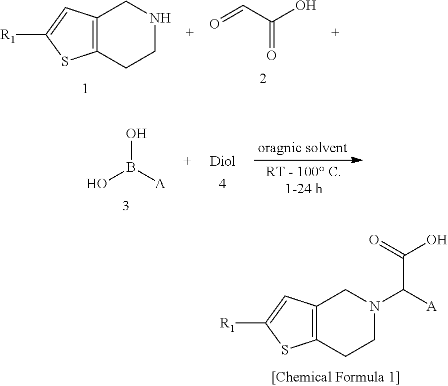
wherein in the Chemical Formula 1,
A is selected from 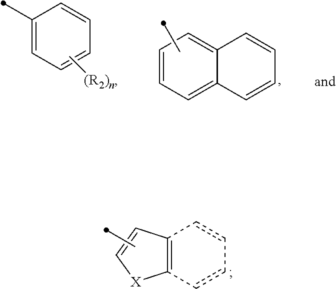
R₁ is hydrogen or an acetyl group;
R₂ is each independently selected from hydrogen, halogen, C₁₋₆ alkyl, C₁₋₆ alkoxy, and nitrile;
n is an integer of 0 to 2; and
dotted lines represent simultaneous absence or presence of bonds.

2. A method for manufacturing a tetrahydrothienopyridine compound of Chemical Formula 1, by a one-pot reaction of multi-component starting materials comprising a tetrahydrothienopyridine compound (1), glyoxylic acid (2), a boronic acid (3), and a molecular sieve (5) as an additive in an organic solvent in a temperature range of room temperature to 100° C. for 1-24 hours: 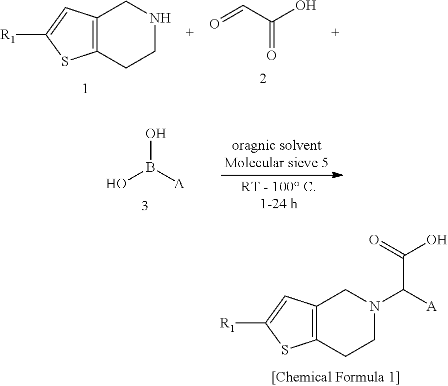
wherein in the Chemical Formula 1,
A is selected from 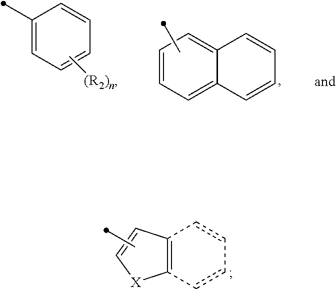
R₁ is hydrogen or an acetyl group;
R₂ is each independently selected from hydrogen, halogen, C₁₋₆ alkyl, C₁₋₆ alkoxy, and nitrile;
n is an integer of 0 to 2; and
dotted lines represent simultaneous absence or presence of bonds.

3. A method for manufacturing a tetrahydrothienopyridine compound of Chemical Formula 2, by a one-pot reaction of multi-component starting materials comprising a tetrahydrothienopyridine compound (1), glyoxylic acid (2), a boronic acid (3), and a diol (4) in an organic solvent in a temperature range of room temperature to 100° C. for 1-24 hours: 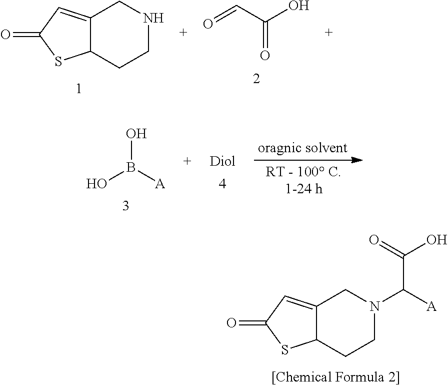
wherein, in the Chemical Formula 2,
A is selected from 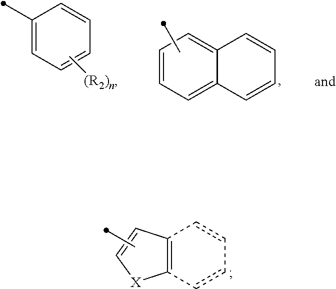
R₂ is each independently hydrogen or halogen;
n is an integer of 0 to 2; and
dotted lines represent simultaneous absence or presence of bonds.

4. A method for manufacturing a tetrahydrothienopyridine compound of Chemical Formula 2, by a one-pot reaction of multi-component starting materials comprising a tetrahydrothienopyridine compound (1), glyoxylic acid (2), a boronic acid (3), and a molecular sieve (5) as an additive in an organic solvent in a temperature range of room temperature to 100° C. for 1-24 hours: 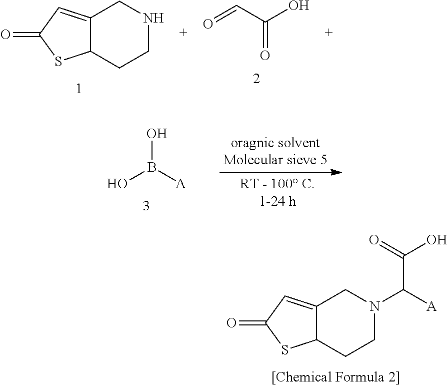
wherein, in the Chemical Formula 2,
A is selected from 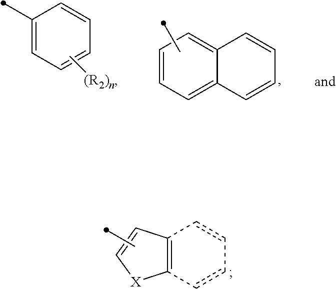
R₂ is each independently hydrogen or halogen;
n is an integer of 0 to 2; and
dotted lines represent simultaneous absence or presence of bonds.

5. The method of claim 1, wherein the organic solvent is at least one selected from the group consisting of DMF, DMSO, CH₂Cl₂, MeOH, EtOH, i-PrOH, THF, Toluene, MeCN, and 1,4-Dioxane.

6. The method of claim 1, wherein the boronic acid (3) is selected from the group consisting of phenyl, 2-fluorophenyl, 2-chlorophenyl, 2-bromophenyl, 2-iodophenyl, 2-methylphenyl, 2-methoxyphenylboronic acid; 3-fluorophenyl, 3-chlorophenyl, 3-bromophenyl, 3-iodophenylboronic acid; 4-fluorophenyl, 4-chlorophenyl, 4-methylphenyl, 4-methoxyphenyl, 4-nitrophenyl, 4-cyanophenylboronic acid; 3-chloro-4-methylphenyl, 3,5-difluorophenyl, 3,4-(methylenedioxy)phenylboronic acid; 1-naphthyl, 2-naphtylboronic acid; 6-fluoro-3-pyridinyl, 6-chloro-3-pyridinyl, 6-bromo-3-pyridinylboronic acid; 2-furyl, 2-thienyl, 2-benzofuryl, 2-benzothienyl, 3-furyl, 3-thienyl, 3-benzofuryl, 3-benzothienylboronic acid; 2-phenylvinyl, 2-(3-fluorophenyl)vinyl, 2-(4-fluorophenyl)vinyl, 2-(4-chlorophenyl)vinylboronic acid.

7. The method of claim 1, wherein the diol (4) is selected from the group consisting of ethylene glycol, 1,3-propanediol, 1,2-cyclopentanediol, 1,2-cycloheanediol, pinacol, and catechol.

8. The method of claim 1, wherein the molecular sieve as an additive is added to a solution resulted from the one-pot, multi-component reaction.

9. The method of claim 2, wherein the molecular sieve is selected from Molecular Sieve 3A, 4A, or 13X.

10. The method of claim 1, wherein in the Chemical Formula 1,
A is 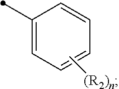
R₁ is hydrogen;
R₂ is each independently selected from the group consisting of hydrogen, fluoro, and chloro; and
n is an integer of 0 to 2.

11. The method of claim 3, wherein in the Chemical Formula 2,
A is 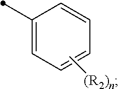
R₂ is independently selected from the group consisting of hydrogen, fluoro, and chloro; and
n is an integer of 0 to 2.

12. The method of claim 2, wherein the organic solvent is at least one selected from the group consisting of DMF, DMSO, CH₂Cl₂, MeOH, EtOH, i-PrOH, THF, Toluene, MeCN, and 1,4-Dioxane.

13. The method of claim 3, wherein the organic solvent is at least one selected from the group consisting of DMF, DMSO, CH₂Cl₂, MeOH, EtOH, i-PrOH, THF, Toluene, MeCN, and 1,4-Dioxane.

14. The method of claim 4, wherein the organic solvent is at least one selected from the group consisting of DMF, DMSO, CH₂Cl₂, MeOH, EtOH, i-PrOH, THF, Toluene, MeCN, and 1,4-Dioxane.

15. The method of claim 2, wherein the boronic acid (3) is selected from the group consisting of phenyl, 2-fluorophenyl, 2-chlorophenyl, 2-bromophenyl, 2-iodophenyl, 2-methylphenyl, 2-methoxyphenylboronic acid; 3-fluorophenyl, 3-chlorophenyl, 3-bromophenyl, 3-iodophenylboronic acid; 4-fluorophenyl, 4-chlorophenyl, 4-methylphenyl, 4-methoxyphenyl, 4-nitrophenyl, 4-cyanophenylboronic acid; 3-chloro-4-methylphenyl, 3,5-difluorophenyl, 3,4-(methylenedioxy)phenylboronic acid; 1-naphthyl, 2-naphtylboronic acid; 6-fluoro-3-pyridinyl, 6-chloro-3-pyridinyl, 6-bromo-3-pyridinylboronic acid; 2-furyl, 2-thienyl, 2-benzofuryl, 2-benzothienyl, 3-furyl, 3-thienyl, 3-benzofuryl, 3-benzothienylboronic acid; 2-phenylvinyl, 2-(3-fluorophenyl)vinyl, 2-(4-fluorophenyl)vinyl, 2-(4-chlorophenyl)vinylboronic acid.

16. The method of claim 3, wherein the boronic acid (3) is selected from the group consisting of phenyl, 2-fluorophenyl, 2-chlorophenyl, 2-bromophenyl, 2-iodophenyl, 2-methylphenyl, 2-methoxyphenylboronic acid; 3-fluorophenyl, 3-chlorophenyl, 3-bromophenyl, 3-iodophenylboronic acid; 4-fluorophenyl, 4-chlorophenyl, 4-methylphenyl, 4-methoxyphenyl, 4-nitrophenyl, 4-cyanophenylboronic acid; 3-chloro-4-methylphenyl, 3,5-difluorophenyl, 3,4-(methylenedioxy)phenylboronic acid; 1-naphthyl, 2-naphtylboronic acid; 6-fluoro-3-pyridinyl, 6-chloro-3-pyridinyl, 6-bromo-3-pyridinylboronic acid; 2-furyl, 2-thienyl, 2-benzofuryl, 2-benzothienyl, 3-furyl, 3-thienyl, 3-benzofuryl, 3-benzothienylboronic acid; 2-phenylvinyl, 2-(3-fluorophenyl)vinyl, 2-(4-fluorophenyl)vinyl, 2-(4-chlorophenyl)vinylboronic acid.

17. The method of claim 4, wherein the boronic acid (3) is selected from the group consisting of phenyl, 2-fluorophenyl, 2-chlorophenyl, 2-bromophenyl, 2-iodophenyl, 2-methylphenyl, 2-methoxyphenylboronic acid; 3-fluorophenyl, 3-chlorophenyl, 3-bromophenyl, 3-iodophenylboronic acid; 4-fluorophenyl, 4-chlorophenyl, 4-methylphenyl, 4-methoxyphenyl, 4-nitrophenyl, 4-cyanophenylboronic acid; 3-chloro-4-methylphenyl, 3,5-difluorophenyl, 3,4-(methylenedioxy)phenylboronic acid; 1-naphthyl, 2-naphtylboronic acid; 6-fluoro-3-pyridinyl, 6-chloro-3-pyridinyl, 6-bromo-3-pyridinylboronic acid; 2-furyl, 2-thienyl, 2-benzofuryl, 2-benzothienyl, 3-furyl, 3-thienyl, 3-benzofuryl, 3-benzothienylboronic acid; 2-phenylvinyl, 2-(3-fluorophenyl)vinyl, 2-(4-fluorophenyl)vinyl, 2-(4-chlorophenyl)vinylboronic acid.

18. The method of claim 2, wherein in the Chemical Formula 1,
A is 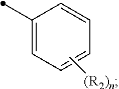
R₁ is hydrogen;
R₂ is each independently selected from the group consisting of hydrogen, fluoro, and chloro; and
n is an integer of 0 to 2.
